(19) United States
(12) Patent Application Publication
(10) Pub. No.: US 2017/0304077 A1
(43) Pub. Date: Oct. 26, 2017

(54) DELIVERY DEVICE FOR GRAFT MATERIAL

(71) Applicant: Medtronic-Xomed, Inc., Jacksonville, FL (US)

(72) Inventors: Louis M. Shadeck, Jacksonville, FL (US); Phillip J. Berman, Jacksonville, FL (US)

(73) Assignee: Medtronic-Xomed, Inc., Jacksonville, FL (US)

(21) Appl. No.: 15/625,190

(22) Filed: Jun. 16, 2017

Related U.S. Application Data
(63) Continuation of application No. 13/871,743, filed on Apr. 26, 2013.

(51) Int. Cl. A61F 2/46; A61B 17/88

(52) U.S. Cl. CPC A61F 2/4601; A61B 17/8816; A61B 17/8822

(57) ABSTRACT

A method of delivering graft material to a surgical site includes positioning graft material in a tube of a delivery device. The tube defines an open end and a bend along its length. A plunger within the tube is advanced to dispense the graft material through the open end.

### CROSS-REFERENCE TO RELATED APPLICATIONS

This application is a continuation of U.S. application Ser. No. 13/871,743, filed Apr. 26, 2013, entitled “Delivery Device for Graft Material”; the entire teachings of which are incorporated herein by reference.

### BACKGROUND

Concepts presented herein relate to delivery of graft material to a target site.

Example applications that utilize graft material include rhinologic functional endoscopic sinus surgery (FESS), spinal, orthopedic and arthroscopic procedures. Regardless of the exact procedure, various instruments for delivery of graft material are currently employed. In some procedures to delivery material to a target site, surrounding structures can be sensitive and thus contact between these structures and the instruments should be avoided. As such, current procedures can be overtly time consuming and require that the instruments pass sensitive structures multiple times to perform the delivery of material. This situation increases the chance of damage to sensitive structures adjacent the target site.

### SUMMARY

A method of delivering graft material to a surgical site includes positioning graft material in a tube of a delivery device. The tube defines an open end and a bend portion along its length. A plunger within the tube is advanced to dispense the graft material through the open end.

A delivery device for delivering graft material to a target site includes a hand piece maintaining an actuator mechanism. A tube that defines a lumen, an open end and a bent portion along its length is coupled to the hand piece. A plunger is positioned within the lumen of the tube and coupled to the actuator mechanism. The actuator mechanism is configured to move the plunger within the lumen from a loading position to an extended position.

### BRIEF DESCRIPTION OF THE DRAWINGS

FIG. 1 is a side view of a delivery device according to a first embodiment.

FIG. 2 is a side view of a delivery device according to a second embodiment.

FIG. 3 is a side view of a distal end of a delivery device.

FIG. 4 is a side view of an alternative plunger of a delivery device.

FIGS. 5 and 6 are schematic views of steps in using a delivery tube to deliver graft material to a cavity.

### DETAILED DESCRIPTION

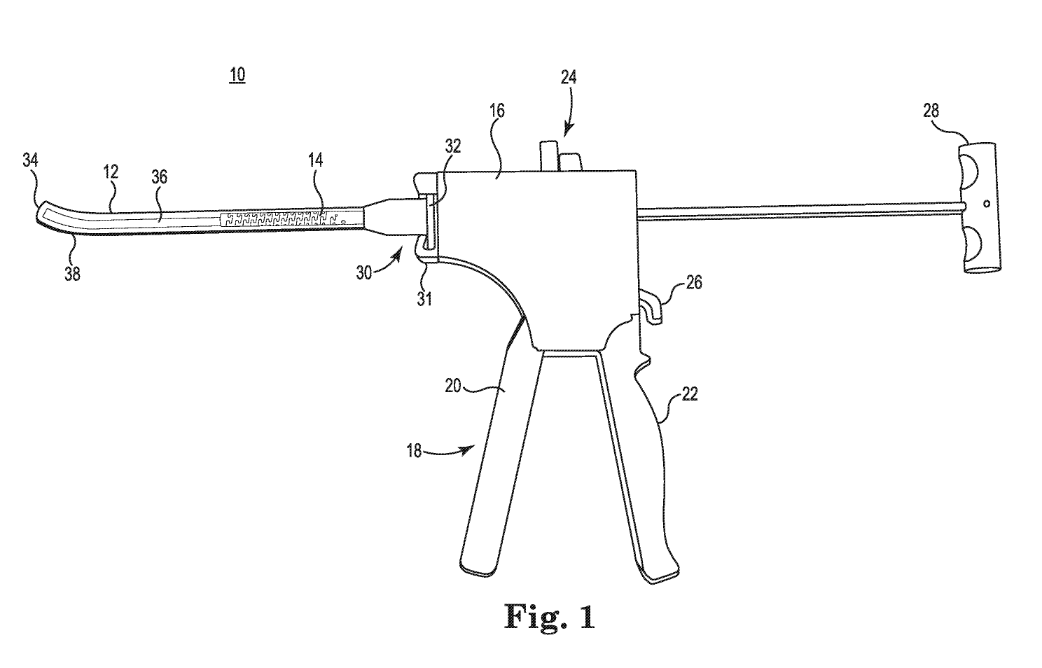

FIG. 1 is a side view of a first embodiment of a delivery device 10. Device 10 includes a delivery tube 12, a plunger 14 positioned within the delivery tube 12 and a hand piece 16 coupled with the tube 12 and plunger 14. Hand piece 16 maintains an actuator mechanism 18 configured to move plunger 14 with respect to tube 12. In one embodiment, the actuator mechanism 18 can move plunger 14 in a controlled, metered manner as desired. In the illustrated embodiment, actuator mechanism 18 includes a first handle 20, a second handle 22 and a ratcheting mechanism generally indicated at 24. As a user squeezes handles 20 and 22 together, ratcheting mechanism 24 moves to operate and advance plunger 14 along the tube 12. Advancement of the plunger 14 can be controlled to advance a predetermined distance each time the actuation mechanism 18 is actuated. This predetermined distance corresponds to a volume of material within the tube that will be dispersed. In a further embodiment, tube 12 can include markings to denote an amount of material positioned with the tube 12.

Hand piece 16 can also include a release mechanism 26 that releases plunger 14 from engagement with the ratchet mechanism 24. In particular, in order to move plunger 14 manually with respect to tube 12, a user can depress the release mechanism 26 and operate a handle 28 to move the plunger 14 to a desired position along tube 12.

Details of the tube 12 are discussed below. In general, however, tube 12 can include a suitable connection mechanism 30 that engages a corresponding receiving mechanism 31 on the hand piece 16 so as to secure the tube 12 to the hand piece 16 during operation of the device 10. Tube 12 includes a first, proximal end 32 and a distal, open end 34. The tube 12 further defines a lumen 36 configured to transport graft material therein. The tube 12 further includes a bend portion 38 positioned between the proximal end 32 and the proximal end 34 and the distal end 34. In one embodiment, a distance from the distal end 34 to the bend portion 38 is approximately in a range from 1.0 to 1.5 inches. Furthermore, the bend portion 38 is defined as having approximately a 4.0 inch radius.

In any event, plunger 14 in FIG. 1 is illustrated in a retracted or loading position that allows graft material to be inserted through distal end 34 and into lumen 36. Once the material is loaded into lumen, plunger 14 can be advanced toward the distal end 34 (i.e., to an extended or delivery position) in order to advance material within the lumen 36 along the tube 12. Ultimately, the material exits the distal end 34.

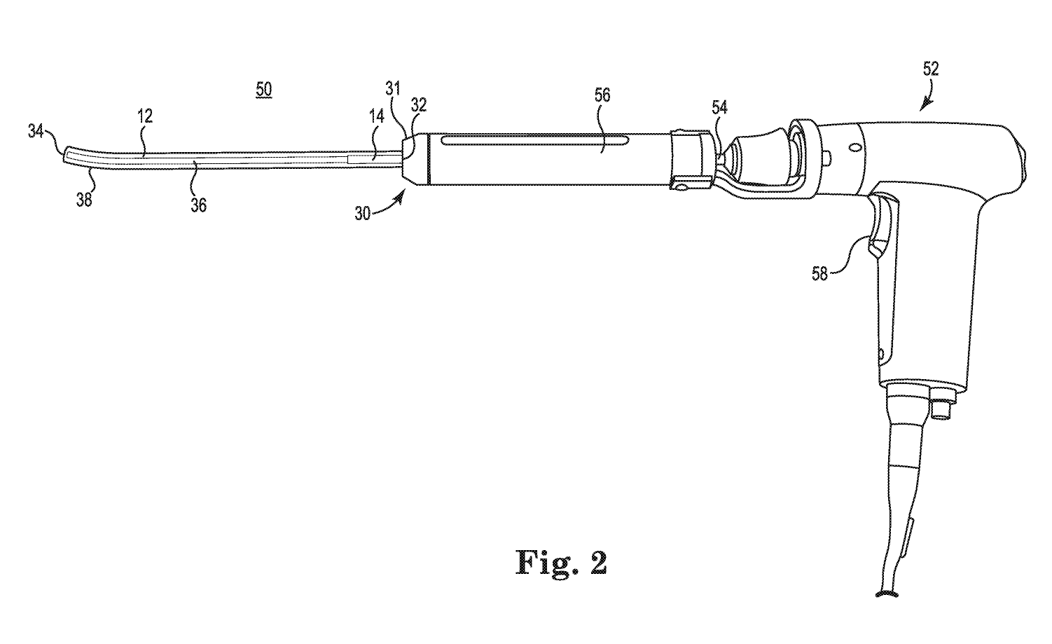

FIG. 2 is a side view of a second embodiment of a delivery device 50 that includes the delivery tube 12 and plunger 14 as discussed above with respect to FIG. 1. As opposed to the hand operated mechanism 18 of device 10, device 50 includes a motorized actuator mechanism 52 that includes a motor to deliver powered rotational movement to a rod 54. A translation mechanism 56 translates the rotational movement of the rod 54 to linear movement so as to move plunger 14 relative to tube 12. In particular, hand piece 52 includes a trigger mechanism 58 that, when depressed, provides rotational power to the rod 54.

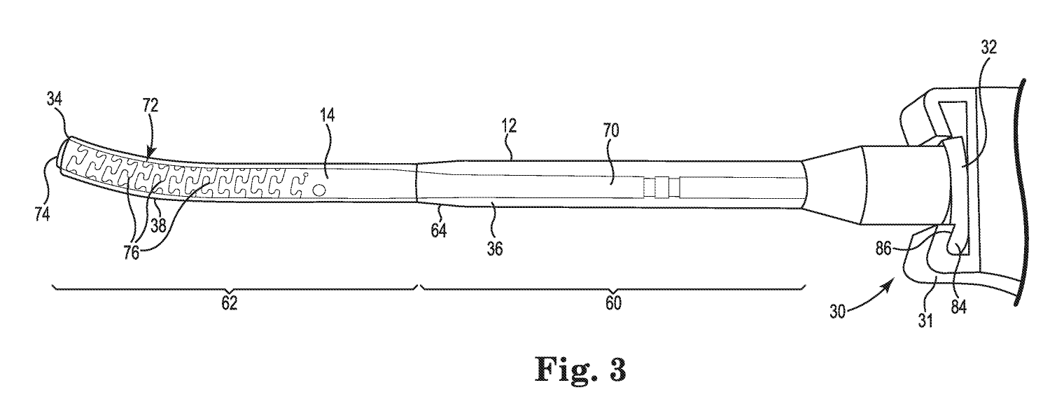

Regardless of a particular actuator mechanism used to position plunger 14 with respect to tube 12, FIG. 3 is a close up view of the tube 12 and plunger 14. As compared with FIG. 1, plunger 14 is illustrated in FIG. 3 in an extended or delivery position, wherein the plunger 14 extends to the distal end 34 of the tube 12. Tube 12 includes a proximal portion 60 and a distal portion 62. The proximal portion 60 extends from the connection mechanism 30 and narrows at a tapered portion 64 to connect with the distal portion 62. Distal portion 62 defines a constant diameter from the tapered portion 64 to the open distal end 34. In one embodiment, an outer diameter of the distal portion is in a range of approximately 5.0-7.0 millimeters. The tapered portion 64 can assist in preventing material from being compacted within the lumen 36.

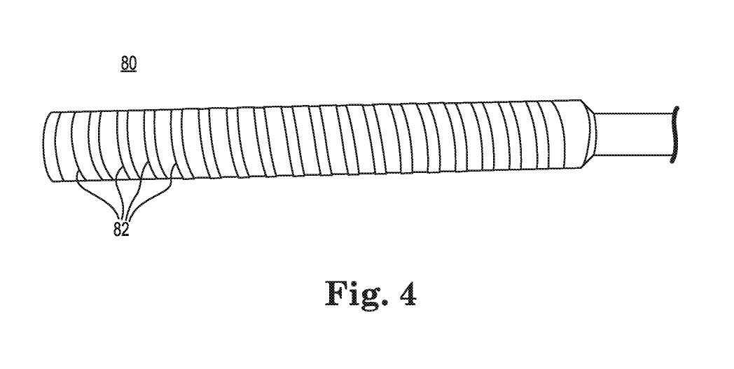

As illustrated, plunger 14 includes a proximal rod 70, a flexible distal portion 72 and a distal tip 74. In one embodiment, the plunger 14 is formed of stainless steel or polyether ether ketone (PEEK). Other materials can be used to form the plunger 14. Prior to use, the plunger 14 can be sterilized. Rod 70 is directly coupled to an actuator mechanism (e.g., mechanism 18 or 52) to move plunger 14 along the tube 12. Flexible distal portion 72 includes a plurality of cuts 76 that impart flexibility within the distal portion 72. In the embodiment illustrated, cuts 76 are dovetail shaped. In alternative embodiments, the cuts 76 can be spiral shaped, double spiral shaped and/or other shapes as desired. For example, FIG. 4 illustrates an alternative plunger 80 that includes double spiral cuts 82 positioned along a length of the plunger 82. Independent of the shape of cuts 76, the cuts 76 impart flexibility on the distal portion 72 so as to allow plunger 14 to move along bend portion 38. Distal tip 74 can be formed of silicon or other surgically safe material as desired.

To secure tube 12 to hand piece 18, the connection mechanism 30 defines a flange 84 positioned at the proximal end 32. Flange 84 is positioned within recesses 86 (one of which is shown) in receiving mechanism 31 of the hand piece 16. To connect connection mechanism 30 to receiving mechanism 31, flange 84 is inserted into receiving mechanism 31 and rotated a quarter turn to position flange 84 within recesses 86.

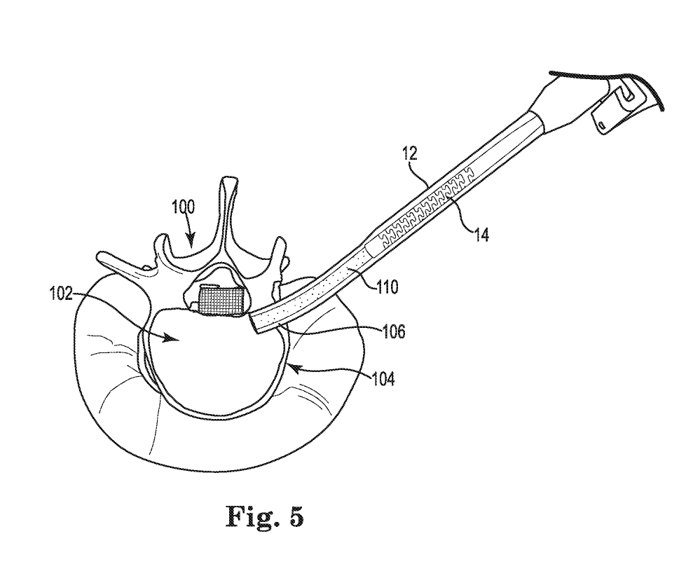

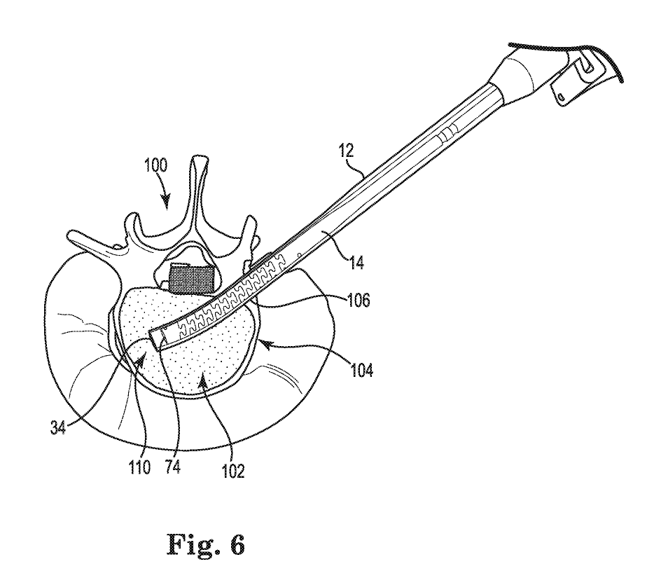

FIGS. 5 and 6 illustrate delivery of material to a cavity at a target site. In the embodiment illustrated, the target site is an intervertebral disc 100. Delivery tube 12 can be modified as desired for delivery of graft material to alternative target sites. For example, bend portion 38 can be positioned at different distances from distal end 34 and/or define a different radius of curvature. By way of reference, the intervertebral disc 100 generally includes a nucleus 102 forming a cavity surrounded by an annulus 104. The tube 12 is employed to deliver graft material 110 positioned within lumen 36 to the nucleus 102. In particular, the plunger 14 is in a loading position (i.e., retracted from distal end 34) and graft material 110 has been positioned within lumen 36. Once the material 110 is loaded, tube 12 approaches the intervertebral disc 100. In one embodiment, the tube 12 is deployed using a transforaminal approach, for example during a transforaminal lumbar interbody fusion (TLIF) procedure. Alternative approaches to the disc 100 are also acceptable and include a posterior approach, posterior-lateral approach, anterior approach, left or right lateral approach, etc.

Regardless of the approach, the tube 12 is positioned through an opening 106 formed in the annulus 104. The opening 106 can be a naturally-occurring tear or similar passage. Alternatively, the opening 106 can be surgically cut or otherwise created in the annulus 104. In FIG. 5, the open distal end 34 of the tube 12 is positioned within the nucleus 102 for delivery of graft material therein. In particular, the plunger 14 is in a retracted position, allowing graft material 110 to be positioned within the lumen 36 distal the tip 74. The distal end 34 is advanced into the nucleus 102 to a desired position. Once positioned within the nucleus 102, as illustrated in FIG. 6, the plunger 14 is distally advanced to an extended position to dispense graft material 110 out the distal end 34 of the delivery tube 12.

Although the present disclosure has been described with reference to preferred embodiments, workers skilled in the art will recognize that changes can be made in form and detail without departing from the spirit and scope of the present disclosure.

What is claimed is:

1. A method of delivering graft material to an intervertebral disc, comprising:
positioning graft material in a tube of a delivery device, wherein the delivery device further includes a first handle and a second handle extending within a first plane; wherein the tube defines an open end and a bend portion at a distal end along a length of the tube; wherein the bed portion extends away from the first and second handles while remaining within the first plane;
delivering the tube to a nucleus of the intervertebral disc; and
advancing a plunger within the tube to dispense the graft material through the open end.
